(19) United States
(12) Patent Application Publication
(10) Pub. No.: US 2017/0224930 A1
(43) Pub. Date: Aug. 10, 2017

(54) SAFETY CONVENIENCE KITS FOR MEDICAL SYRINGES

(71) Applicant: Allison Medical, Inc., Littleton, CO (US)

(72) Inventors: Kendall Patterick Thorne, Layton, UT (US); Gale Harrison Thorne, JR., Bountiful, UT (US); Gale Harrison Thorne, Bountiful, UT (US)

(73) Assignee: Allison Medical, Inc., Littleton, CO (US)

(21) Appl. No.: 15/015,379

(22) Filed: Feb. 4, 2016

(51) Int. Cl. A61M 5/32; B65D 25/04; B65D 43/16; A61M 5/00

(52) U.S. Cl. CPC A61M 5/321; A61M 5/002; B65D 25/04; B65D 43/16

(57) ABSTRACT

A convenience kit which is usable in an ambulatory environment for bagged products which have exposed and potentially contaminated sharps as an end product. Preferred use focus of the kit is for bagged syringes. The kit provides accessible sections for accessing the unused bagged parts and for storing excess parts once a bag is opened. A removable protective cover is provided for the unused product storage. A single step used parts disposal system comprises an elongated orifice and a pair of opposing chutes which are constructed to provide a one-way path into a containment compartment which is expanded in volume as new products are removed. As the kit may be used in an ambulatory environment permissive to rotational and vibratory forces, the upper chute is pivotally affixed to rotate and close the elongated orifice by the same forces which urge products within the compartment toward the orifice.

### FIELD OF INVENTION

This invention relates generally to kits and containers for transport, storage and access prior to use and to containment safety of syringes after use. More broadly, it is particularly related to devices used for safety to enclose medical needles and other sharps after use. It is also particularly related to kits and containers which provide for delivery of packages, each containing a plurality of products (e.g. syringes) which may be opened in a field environment for single use syringe applications leaving remaining unused products resident in an opened package.

### DESCRIPTION OF RELATED ART

U.S. Pat. No. 6,685,017 filed Jan. 14, 2002, and issued Feb. 3, 2004, to Charles W. Erickson (Erickson) discloses and claims a multifunction box for transporting, storing and accessing unused syringes and for receiving and storing used syringes. The box of Erickson comprises a dividing tray which is impelled by gravity to lower and open a cavity for used syringes which are inserted through a multi-step syringe feed means for receiving used syringes after new syringes are withdrawn from the box. The box of Erickson is taught to have an opening at the bottom for individually accessing and withdrawing bags of syringes.

U.S. Pat. No. 5,184,720 filed Nov. 8, 1991, and issued Feb. 9, 1993 to Gilbert Packer, et al. (Packer) discloses a used syringe receptacle having two elongated apertures and associated chutes through which used syringes are displaced into a safety container. The apertures and chutes comprise no moving parts and are geometrically disposed to preclude a sharp cannula from being exposed outside the apertures of a stationary, upright container. However, there is no method or structure for assuring shaking or other displacement at the receptacle site could not somehow permit a previously inserted syringe from being displaced there thru.

In broad context, boxes and receptacles for transporting, storing and accessing new syringes and for subsequently collecting and storing used syringes are presently known in commerce.

### BRIEF SUMMARY AND OBJECTS OF THE INVENTION

In brief summary, this novel invention alleviates all of the known problems related to providing a kit for transporting, storing and providing new products (e.g. syringes) from an unused product enclosing container and to disposing of used products (with exposed sharps) with convenience and safety in an ambulatory environment. While application for the kit can be broad, focus is upon a kit for packaged syringes. For this reason, disclosure of the instant invention is focused on packaged syringes even though one skilled in packaging and sharps container art can readily envision wider applications for the kit.

While syringes, such as those used in treatment of diabetes, are designed and capped to be widely used by both practitioners and those afflicted in a broad variety of circumstances, it is known to be good practice to maintain all such devices in as clean surroundings as possible. Further, previously opened transport cartons or packages of syringes (or other products) may be disposed in unclean places like car trunks, hotel rooms, barns, etc. in the process of apportioning and using residual syringes from a large boxed supply.

This instant invention affords a convenient kit whereby new syringes are facilely accessed and stored enclosed preparatory to use and are disposed of after use. Basic to the kit are three container parts:

1. a bottom part which provides disparate storage compartments for each of new syringes and of used syringes,

2. a top part which is securely affixed to the bottom part and which provides a protective accessible cover about a section for new syringes and an elongated aperture through which used syringes are simply dropped for disposal safety, and

3. a tray part which is disposed between the top and bottom parts to provide a convenient access orifice to new syringes and a storage tray for retrieved syringes planned for near term use.

Recognizing that such kits are products for an ambulatory application and that, in such applications, a predetermined orientation relative to gravity is not always practical, storage, transport and product access are designed in the instant invention to be relatively orientation independent. As an example, as syringes are removed from the bottom for use, a compartment separation plate, disposed in the bottom part, is automatically impelled (independent of kit orientation relative to gravity) to increase compartment capacity for storage of used syringes and to displace new product to an access orifice in the tray part.

Also facile used syringe disposal is important in ambulatory settings. Due to a wide variety of circumstances associated with syringe use, requirement for only dropping a syringe by a single hand step is considered far superior to added complication for syringe disposition requiring multiple steps. In this instant invention, the elongated aperture provides entry to a tortuous one-way passage provided by two opposing chutes disposed above the used syringe compartment.

While the opposing chutes are effective in countering effluent displacement of used syringes from the used syringe compartment, it is possible that continuous shaking and/or carton rotation could result in some contents spilling out of such a pathway. For this reason, the upper chute is hinged and disproportionately weighted to balance to an open state for used syringe disposal when the kit is upright and static, and to rotate to a closed state when acted upon by the same forces which would displace items from used syringe compartment.

Of course, such a convenience kit is only complete and ready for use when new syringes are first stored in the new syringe compartment. While such syringes may be so stored individually, it is currently preferred to store bags of multiple syringes. In such a case, acquisition and use of a single syringe results, generally, in a residual number of unused syringes kept out of the bottom part for later use. For this purpose, the tray part comprises space for storing the residual syringes under protective cover of the top part.

Once, all of the new syringes are used and returned to the used syringe compartment, it is likely desirable to close and lock the kit container from future use. For this purpose, access is provided to a locking member which rotates and locks and secures the hinged chute in a closed state for safety.

Impellers, which provide force for displacing the compartment separation plate may be stretched elastic bands, springs or other memory based energy impelling items. If elastic bands are used, material for the bands must be sufficiently elastic to withstand long periods of being stretched without losing effective elasticity. Such bands are currently available commercially.

A latch is provided for the displaceable section of the top whereby products under the top (i.e. those atop the tray and those at the access orifice) are protectively covered.

Accordingly, it is a primary object to provide a safety convenience kit for transporting, storing and accessing new syringes and a safety enclosure for used syringes.

It is also a primary object to provide a single hand, single step design and procedure for depositing used syringes into a safety storage and retention chamber.

It is an important object to provide a self-closing structure associated with used syringe deposit which assures complete retention of deposited items independent of kit orientation and movement.

It is an object to provide a common, but disparately divided, bottom for the kit which provides a first chamber storage space for unused items, which is reduced in volume as items are removed therefrom, and a second chamber for secure containment of used syringes, the volume of the second chamber spontaneously increasing as the contents of the first chamber are removed.

It is a very important object that volume reduction of the first chamber storage compartment be reflexive and occur independent of kit orientation, relative to direction of force of gravity.

It is a consequential object that facility is provided for covering all unused product whether yet contained in the bottom part or disposed on the tray part.

It is an object to provide an interlock which closes and locks the used syringe entry aperture and blocks further use of the convenience kit.

These and other objects and features of the present invention will be apparent from the detailed description taken with reference to accompanying drawings.

### BRIEF DESCRIPTION OF THE DRAWINGS

FIG. 1 is a perspective of a closed convenience kit according to the instant invention.

FIG. 2 is a cross section of a side elevation of the kit seen in FIG. 1.

FIG. 2A is a cross section like FIG. 2, but inverted to permit hinged parts to rotate.

FIG. 3 is an exploded perspective of the kit seen in FIG. 1.

FIG. 3A is an inverted perspective of a tray part of the kit seen in FIG. 3.

FIG. 4 is an exploded side elevation of the kit seen in FIG. 1.

FIG. 5 is a perspective of the kit seen in FIG. 1 with syringes removed from a bottom part resulting in a single syringe being disposed for ready accessing on a portion of a tray part.

FIG. 6 is a side elevation cross section similar to the cross section of FIG. 2, but with one bag (of syringes) removed and one syringe disposed on the tray part.

FIG. 7 is a perspective of an upper chute component used in the formation of a tortuous path for disposing used syringes.

FIG. 8 is a perspective of a dividing plate used to maintain used and new syringes disparate and to displace packages of new syringes toward an access orifice as individual syringe packages are displaced from the bottom part.

FIG. 9 is a top elevation of an unfilled bottom part whereby an impelling elastic band is affixed between the bottom part and a dividing plate.

FIG. 10 is a perspective showing a syringe cap displaced from an opening in a top part of the kit seen in FIG. 1.

FIG. 11 is a perspective in which the syringe cap seen in FIG. 10 is displaced into the opening in top part of the kit.

FIG. 12 is a side elevation of the kit seen in FIG. 11.

### DETAILED DESCRIPTION OF THE ILLUSTRATED EMBODIMENTS

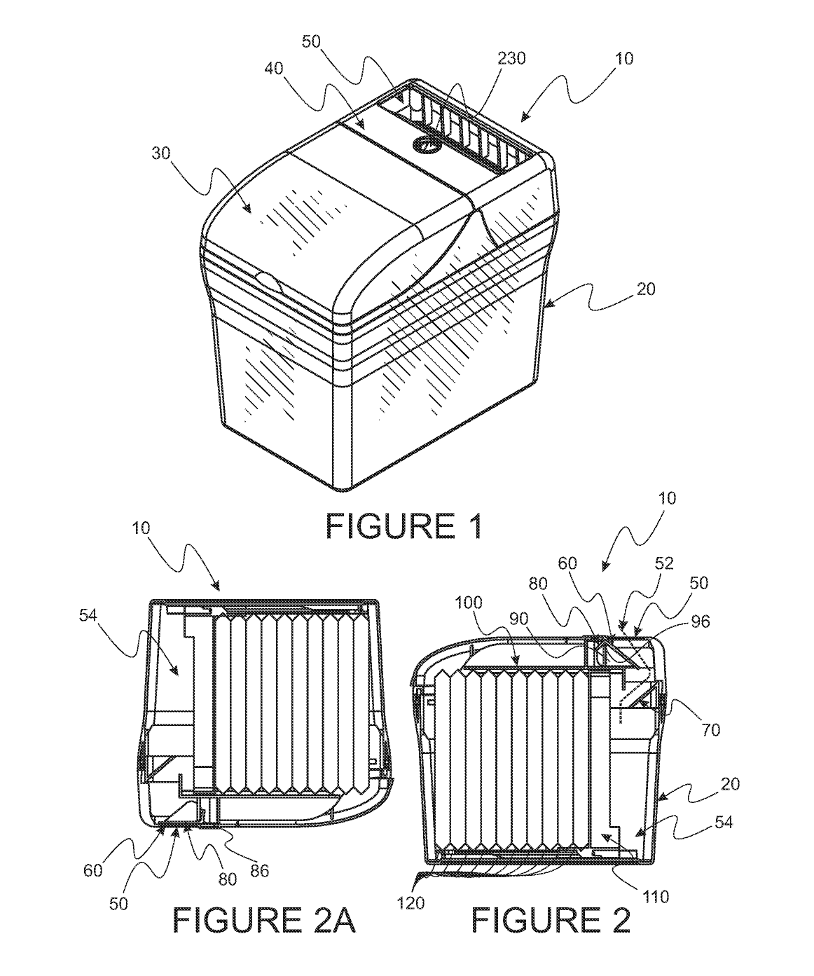

In this description, the term proximal is used to indicate the segment of the device normally closest to the object of the sentence describing its position. The term distal refers to the other end. Reference is now made to the embodiments illustrated in FIGS. 1-12 wherein like numerals are used to designate like parts throughout. Primes of numbers are used for indicating similar but not exact replications of like numbered parts.

A convenience kit 10 made according to the instant invention is seen in FIG. 1. Kit 10 comprises a bottom part 20 and a top part 30. Top part 30 comprises a used syringe receiving section 40 which is securely affixed to bottom part 20. Within section 40, top part 30 further comprises an elongated orifice 50 sized and shaped for facile, drop-through deposition of a used syringe.

As seen in FIG. 2, a disposal pathway (indicated by dashed line 52) is a tortuous pathway defined by an upper chute 60 and a lower chute 70. Pathway 52 is designed as a relatively unobstructed course for depositing used syringes and to be an obstruction to retained syringes already contained in a used syringe compartment 54 of bottom part 20 as long as kit 10 is upright and static, as is well understood by those skilled in sharps collector art.

However, for kit 10 to be completely effective as a container for used syringes in a range of ambulatory environments in which kit 10 may be rotated and/or shaken, it is not sufficient to rely on a tortuous pathway alone for there remains a likelihood that one or more captured syringes could be ejected outward via pathway 52 through orifice 50 as a result of severe vibration and rotation, conditions which may be experienced in the ambulatory environment.

For this reason, chute 60 is formed as a portion of a pivotal component 80. Component 80 is better seen in FIG. 7 to comprise chute 60, an angled lever 82, catch tabs (commonly numbered 84) and a top crease 86. Angled lever 82 comprises a first stop 88, a fulcrum interface 90 and a second stop 92. A second angled lever 82′ comprising similar parts is disposed on the opposite side 94 of component 80. Referring once more to FIG. 2, kit 10 comprises a rib 96 disposed under interface 90. Weight (mass accelerated by gravity) of chute 60 is sufficiently great to displace chute 60 (when disposed upon rib 96) to an open state as seen in FIG. 2.

However, if kit 10 is reoriented from the upright state seen in FIG. 2, to another state such as that seen, by example in FIG. 2A, component 80 is also reoriented due to rotation and gravity to cause chute 60 to close orifice 50. Interestingly, all vibration and rotational forces which drive used product toward orifice 50 also act to close, at least partially, chute 60 to obstruct inadvertent ejection of objects contained in compartment 54, thereby. Note also, that when kit 10 is so inverted that rotation of component 80 is about crease 86.

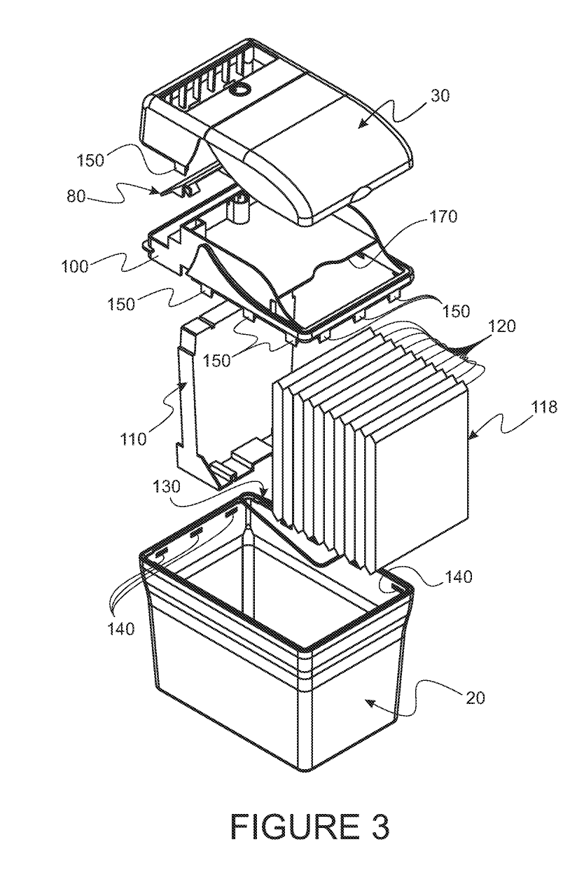

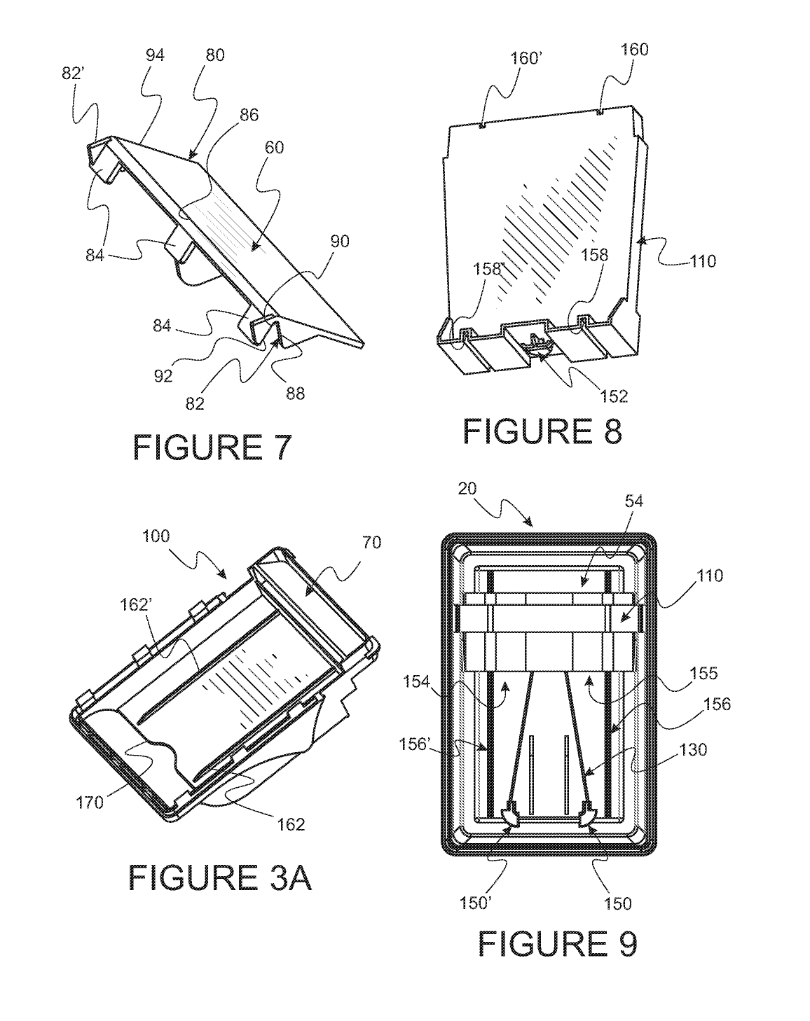

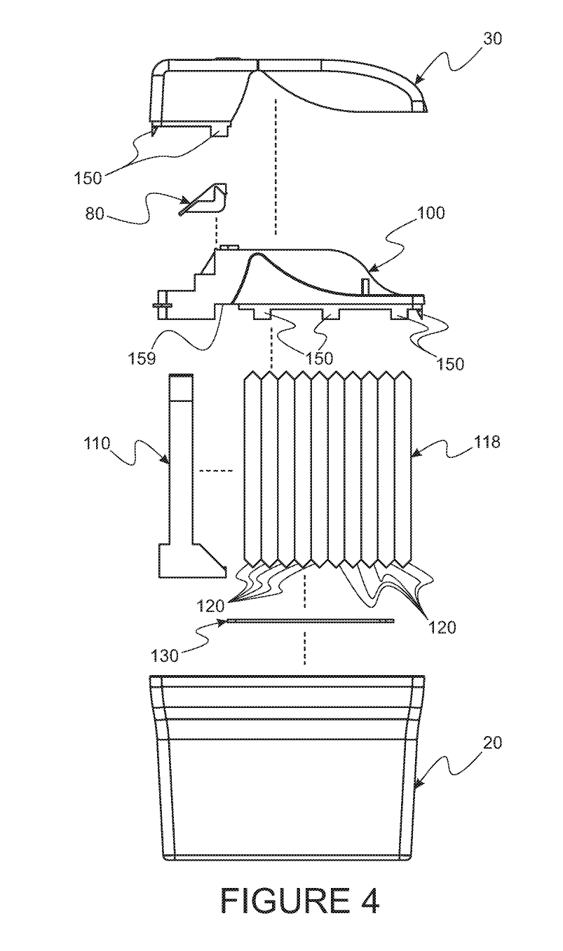

Exploded views of parts and components of kit 10 are seen in FIGS. 3 and 4. A completely assembled and filled kit 10 comprises top part 30, component 80, a tray part 100, a compartment dividing plate 110, a stack 118 of bagged syringes (each generally numbered 120), an elastic band 130 and bottom part 20.

Bottom part 20 comprises a plurality of slots, generally numbered 140 for securely affixing bottom part 20 to tray part 100 and top part 30 (as seen in FIG. 3). Slots 140 and associated connecting tabs, generally numbered 150, on parts 30 and 100 are provided as part of known contemporary injection molding technology for securely affixing parts 30 and 100 to bottom part 20. Also top part 30, component 80, tray part 100, compartment dividing plate 110, and bottom part 20 are all preferably made by injection molding.

Elastic band 130 should be made from elastic material which, when stretched, does not greatly degrade over time. An example of such material is Promostretch Band, available from Aero Rubber Company, Tinley Park, Ill. Of course, bagged syringes 120 are well known and widely commercially available.

Referring to FIG. 9, bottom part 20 is seen with dividing plate 110 displaced to show an elastic band 130 affixed by latch pegs 150 and 150′ to bottom part 20. Not seen in FIG. 9 is another latch peg 152 provided for affixing band 130 to dividing plate 110. Peg 152 is best seen in FIG. 8, molded to the bottom of plate 110. When so affixed, band 130 becomes an effective impeller to displace plate 110 and syringe bags 120 to decrease the volume of new syringe compartment 154 and thereby increase used syringe compartment 54 as bagged syringes 120 are accessed from bottom part 20. Bags 120 are accessed for syringe use through an orifice associated with tray part 100, disclosed in detail hereafter.

Of course, plate 110 must not only be impelled to assure bag displacement, but also be kept upright and orthogonally disposed relative to bottom part 20 inner bottom surface 155 to assure stable geometry of compartments 54 and 154. For this purpose, rails 156 and 156′ provide a stabilizing track for plate 110 within slots 158 and 158′, seen in FIGS. 9 and 8, respectively. Tray part 100 should be disposed to contain and maintain plate 110 in an orthogonal state, as well, as seen in FIG. 2.

Also, for such a purpose, rails on the bottom 159 of tray part 100 are provided for top slots 160 and 160′, seen in FIG. 8. An exemplary rail 162 is seen in FIG. 6. Both rails 162 and 162′ are seen in a bottom portion of tray part 100 in FIG. 3A. In combination, rails 156, 156′, 162 and 162′ act in conjunction with associated slots 158, 158′, 160 and 160′, respectively disposed in plate 110, to maintain orthoogonal stability of plate 110. Such is especially the case where ambulatory motion applies rotational and vibratory forces to kit 10. To further stabilize plate 110, rails 158, 158′, 160 and 160′ may comprise side borne ratchets with associated pawls molded into one or more slots 158, 158′, 160 and 160′ to provide one-way stops as plate 110 is displaced.

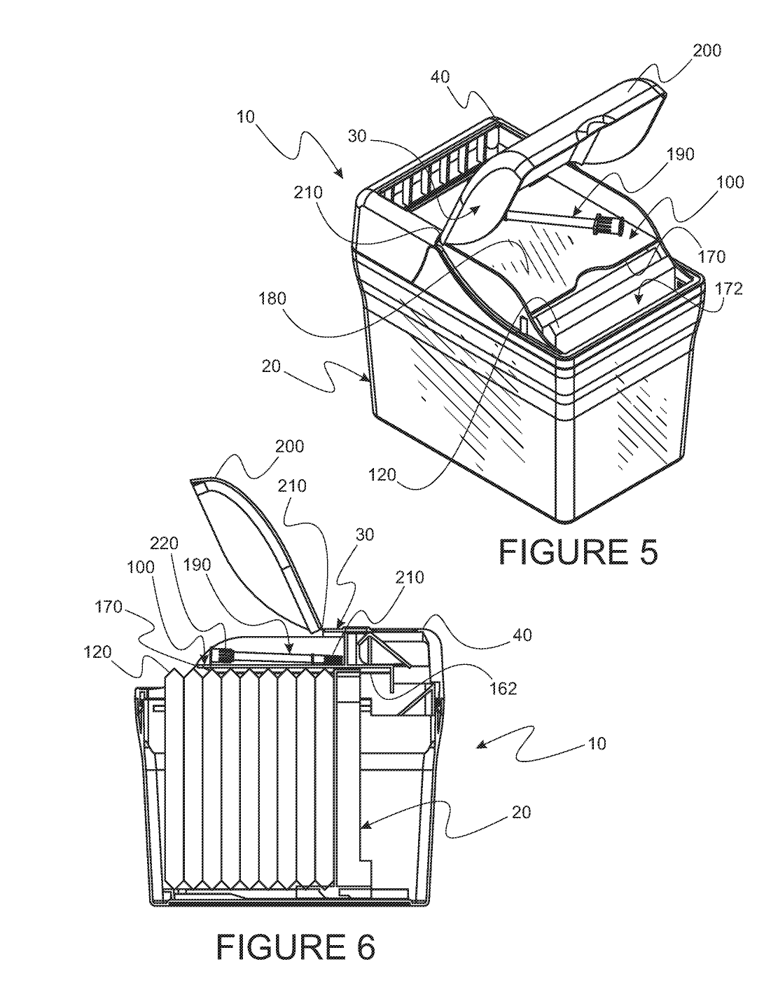

Tray part 100, seen individually in FIGS. 3, 3A and 4 is securely affixed to bottom part 20 and shortened at a fore-end edge 170 (see FIG. 3) to provide an access orifice 172 to syringe bags 120 disposed in bottom 20 (as best seen in FIG. 5). Distal from edge 170 tray part 100 is constructed to provide a shelf 180. As seen in FIGS. 5 and 6, shelf 180 is sized and shaped to provide a storage compartment for syringes remaining unused from a bag 120 accessed from bottom 20. As an example, ten unused syringes, each numbered 190, may be contained in a bag 120 of syringes. A single exemplary syringe 190 is disposed on shelf 180 in each of FIGS. 5 and 6, although space allocation should be large enough for contents of a complete bag 120 of syringes. Also as seen in FIGS. 5 and 6, top part 30 comprises a closeable cover 200 which is closed to cover and protect unused syringes through storage and transport until time for use.

Top part 30 cover 200 is pivotally affixed to section 40 preferably via a living hinge 210 to permit access to contents of shelf 180 and orifice 172. Further, when closed, cover 200 comprises a latch (not shown) which provides assurance of a protective environment for transport and storage. Such latches are well known in compartment closures in plastic container art.

Attention is now drawn to the single syringe 190 seen in FIG. 6. For protective sterility, syringe 190 comprises a cap 210 for protection at a sharpened cannula end and another cap 220 for the other end. Both caps are removed and usually thrown away at the time of syringe use. Of course, once used, the uncapped syringe 190 is dropped through orifice 50 (see FIG. 2) for disposal.

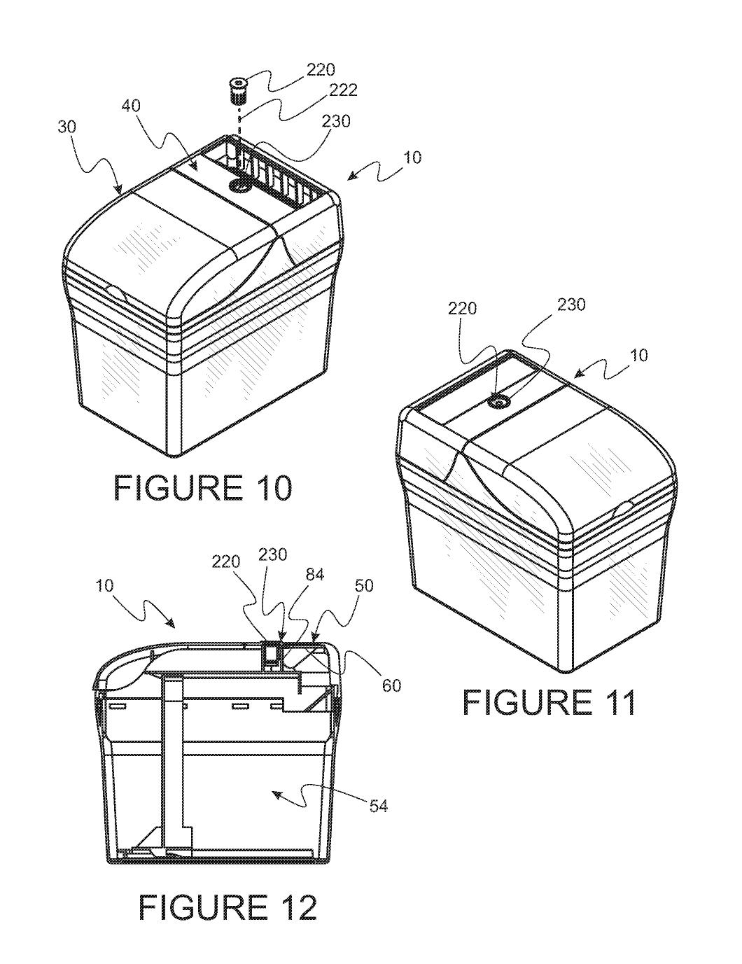

Referring to FIGS. 10-12, section 40 of top part 30 further comprises a medially disposed hole 230 (also seen in FIG. 1). Displaced above hole 230 along dashed line 222, in FIG. 10, is a cap 220, likely removed from a last unused syringe 190 available from kit 10. For once kit 10 is emptied of unused syringes 190 and all used syringes 190 are deposited into compartment 54, see FIGS. 2 and 12, kit 10 is securely closed and further use and access terminated by displacing a cap 220 into hole 230. A lock is formed by geometric communication between hole 230 and a tab 84 (see FIG. 7) of component 80 such that when cap 220 is inserted through hole 230 pivotal contact is made with a tab 84. When cap 220 is fully inserted, as seen in FIGS. 11 and 12, tab 84 is rotated to a vertical state and associated chute 60 is rotated to close orifice 50. It is preferred that hole 230 and cap 220 be so engaged that removal of cap 220 cannot be achieved without a tool. Also, it is preferable that like parts for kits 10 be able to “nested” for efficiency of shipping before assembly.

The invention may be embodied in other specific forms for syringes and for other sharps products without departing from the spirit or essential characteristics thereof. The present embodiment is therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed and desired to be secured by Letters Patent is:

1. An angle of orientation independent convenience kit for transporting, storing and accessing new syringes and for a safety enclosure for used syringes, said kit comprising:
a container further comprising:
a. a bottom part comprising two disparate compartments comprising a first compartment from which new syringes are accessed and a second compartment for receiving and retaining used syringes;
b. a top part securely affixed to said bottom part, said top part comprising a cover which, in a first section, is displaceable from a covering state for accessing new syringes and which, in a second section, comprising an elongated aperture through which used syringes are displaced for disposal safety within said second compartment;
c. a tray part securely disposed between said bottom part and said top part, said tray part being disposed in communication with said first compartment and said first section and further comprising access to an orifice through which new syringes can be stored in said first compartment are displaced for use;
d. used syringe dispensing chutes which in cooperation with said elongated aperture provides a single direction pathway for deposition and secure storage of used syringes, said chutes comprising (1) a hinged ramp which provides for uninhibited passage when disposed in an open state, but which is rotated to a closed state by the same forces which act to displace items from said second compartment, to thereby assure retention of all items resident in the second compartment and (2) a second ramp disposed, in cooperation with said hinged ramp, to provide a tortuous pathway whereby disposing of each syringe involves but a single step of dropping a syringe through the elongated aperture; and
e. a compartment separation system comprising a compartment dividing plate and a memory based impeller for urging said plate to be displaced, independent of angle of kit orientation relative to gravity, to increase the size of the second compartment when a portion of the first compartment is evacuated by acquisition of syringe products therefrom.

2. A convenience kit according to claim 1 wherein said tray part comprises space having capacity for accruing a plurality of new syringes displaced from said first compartment.

3. A convenience kit according to claim 1 wherein said tray part comprises at least one syringe disposed thereon.

4. A convenience kit according to claim 1 wherein said kit comprises syringes.

5. A convenience kit according to claim 1 wherein said first compartment comprises at least one bag comprising a plurality of syringes.

6. A convenience kit according to claim 1 wherein said second ramp is securely affixed to said tray part.

7. A convenience kit according to claim 1 wherein said top part comprises an access orifice where through insertion of a locking part closes and locks said hinged ramp in a closed state.

8. A convenience kit according to claim 1 wherein said top part comprises a living hinge whereby said first section is opened for access.

9. A convenience kit according to claim 1 wherein said impeller comprises a stretched elastic band.

10. A convenience kit according to claim 9 wherein said elastic band comprises elastic material which retains elastic properties while stretched for an extended period of time.

11. A convenience kit according to claim 1 wherein said displaceable section in cooperation with said bottom comprises a releasable latch whereby said section is held closed against said bottom until manually displaced.

12. An angle of orientation independent convenience kit for transporting, storing and accessing new bagged sharps products and for a safety enclosure for used sharps of the products, said kit comprising:
a container further comprising:
a. a bottom part comprising two disparate compartments comprising a first compartment from which new bagged sharps products are accessed and a second compartment for receiving and retaining used and contaminated sharps;
b. a top part securely affixed to said bottom part, said top part comprising a cover which, in a first section, is displaceable from a covering state for accessing new bagged products and which, in a second section, comprising an elongated aperture through which used and contaminated sharps are displaced for disposal safety within said second compartment;
c. a tray part securely disposed between said bottom part and said top part, said tray part being disposed in communication with said first compartment and said first section and further comprising an orifice through which new bagged products stored in said first compartment are displaced for use;
d. used and contaminated product dispensing chutes which in cooperation with said elongated aperture provides a single direction pathway for deposition and secure storage of used and contaminated sharps, said chutes comprising (1) a pivotal ramp which provides for uninhibited passage when disposed in an open state, but which is rotated to a closed state by the same forces which act to displace items from said second compartment, to thereby assure retention of all items resident in the second compartment and (2) a second ramp disposed, in cooperation with said pitotal ramp, to provide a tortuous pathway whereby disposing of each used and contaminated product involves but a single step of dropping a syringe through the elongated aperture; and
e. a compartment separation system comprising a compartment dividing plate and a memory based impeller for urging said plate to be displaced, independent of angle of kit orientation relative to gravity, to increase the size of the second compartment when a portion of the first compartment is evacuated by acquisition of products therefrom.
